(19) United States
(12) Patent Application Publication
(10) Pub. No.: US 2018/0353324 A1
(43) Pub. Date: Dec. 13, 2018

(54) THERMAL CONTROL OF LIQUIDS FOR TRANSCUTANEOUS DELIVERY

(71) Applicant: Undoit Medical LTD, Yoqneam (IL)

(72) Inventors: Gershon Goldenberg, Pardes Hanna Karkur (IL); Israel Birenbaum, Zichron Yaacov (IL); Eran Krieger, Ein Ayala (IL)

(21) Appl. No.: 15/779,964

(22) PCT Filed: Nov. 30, 2016
(86) PCT No.: PCT/IL2016/051281

§ 371 Date: May 30, 2018

(30) Foreign Application Priority Data
Dec. 3, 2015 (IL) 242942

(51) Int. Cl. A61F 7/00

(52) U.S. Cl. CPC A61F 7/0085; A61F 7/007; A61F 2007/0052; A61F 2007/0095; A61F 2007/0076; A61F 2007/0087; A61F 2007/0093; A61F 2007/0069

(57) ABSTRACT

The invention provides a device for controlling the temperature of liquids delivered to the skin during cosmetic, diagnostic, or curative procedures. The invention further provides procedures for treating the skin, including an efficient procedure for removing pigments or tattoos, employing a liquid delivered to the skin with precisely controlled temperature and flowrate.

### FIELD OF THE INVENTION

The present invention relates to a device for controlling the temperature of liquids delivered to the skin or into the skin during various treatments, including cosmetic, aesthetic, diagnostic, or curative procedures, inter alia procedures for removing pigments and tattoos.

### BACKGROUND OF THE INVENTION

Transcutaneous transport of materials is important in cosmetic, aesthetic, diagnostic and curative methods. Materials may be applied onto the skin or intradermally to various depths, depending on the used technique and desired effects. Local effects are needed in some applications, and systemic effects in others. The transported agents may include local anesthesia, diagnostic agents, curative agents, and various other reagents. Many devices have been developed for transdermal (transcutaneous) administration of various agents, usually in a liquid form. It is very important, and sometimes critical, to precisely dose the agents and to accurately manage the delivery process, while assuring the desired effective concentration of the agents in situ; to that end, the temperature of the delivered liquid should be precisely controlled in some applications, because temperature strongly affects the reaction rate and the diffusion process, as well as partitioning. As the liquid stock may be located at undefined temperatures and distances, the temperature of the transcutaneously delivered agents is often difficult to control, particularly when the tip or other delivery member in contact with the skin is disposable. It is therefore an object of this invention to provide a system for the temperature control of transcutaneously delivered agents.

According to some reports, more than one third of America's young adults have at least one tattoo today, and as people quickly change their mind about what is or is not “cool”, the number of people wishing to have a tattoo removed from their skin also increases. However, the tattoo pigment is an integral part of the dermis cells and its removal is difficult and painful. WO 2012/40643 disclosed a highly efficient needling technology based on skin puncturing by micro needles, capable to remove extremely large tattoos, and even multiple-color tattoos which cannot be removed by conventional laser techniques. The technology, known as Trigation™ technology, includes the steps of applying onto the skin a sterile active fluid via a delivery tip, puncturing the skin with a microneedle array, and sucking the internal ingredients and used fluid out of the skin. Improvements of tattoo-removing methods aim at more complete removal of the tattoo pigments from the skin, at lowering the invasiveness of the process and reducing the treatment time, and at reducing the scar formation and healing time. Trigation™ technology is particularly efficient in all these respects, but its full potential might be still better utilized if the temperature of said active liquid might be precisely controlled, because the temperature of said liquid strongly affects all physical, chemical, and biological processes inside the dermis, where the liquid is pushed by the micro needle array shortly after its release on the skin from said delivery tip. The stock solution of said active liquid is located in an infusion bag at room temperature and flows to the skin via a hand piece without temperature control. It is therefore another object of this invention to provide a system and a device for the temperature control of active liquids for transcutaneous use which are employed in tattoo removal.

It is still another object of this invention to provide a device for temperature control of a liquid delivered onto the skin or into the skin for cosmetic, aesthetic, diagnostic, or curative procedure.

It is a further object of this invention to provide a method for controlling the temperature of a liquid delivered onto the skin or into the skin for cosmetic, aesthetic, diagnostic, or curative procedure.

It is a still further object of this invention to provide a system for controlling the temperature of a liquid delivered onto the skin or into the skin for cosmetic, aesthetic, diagnostic, or curative procedure.

This invention also aims at providing a device for adjusting the temperature of a liquid delivered onto the skin or into the skin for a cosmetic, aesthetic, diagnostic, or curative procedure, or for a skin treatment.

It is also an object of this invention to provide a device for adjusting the temperature of a liquid delivered onto the skin for tattoo removal.

It is also another object of this invention to provide a system and method for controlling the temperature of a liquid delivered onto the skin for removing a tattoo or internal pigments, including an extremely large tattoo or a multicolor tattoo.

Other objects and advantages of present invention will appear as description proceeds.

### SUMMARY OF THE INVENTION

The present invention provides a device and a system for controlling the temperature of a liquid delivered onto or into the skin of a subject, comprising i) a container for storing said liquid at a temperature between about 0° C. and ambient temperature; ii) a sufficiently precise liquid pump, such as a peristaltic pump conferring a predetermined flowrate to said liquid; iii) a heat exchanger which heats or cools the liquid, conferring a predetermined temperature of up to 60° C., such as up to 50° C. or up to 45° C., usually between 5° C. and 40° C. to said liquid (working temperature); iv) a temperature sensor detecting the temperature of the liquid before leaving said heat exchanger; v) a heat pump, such as a solid-state Peltier module in good thermal contact with said heat exchanger on one side and with a fan and cooling heat sink on the other side; vi) a tip or other delivery member in contact with said skin and irrigating the skin with said liquid; vii) a tubing delivering said liquid from said container to said tip; viii) a microprocessor receiving the temperature from said sensor and activating said heat pump; said device providing a working liquid at a predetermined flowrate and temperature for a cosmetic, aesthetic, diagnostic, or curative procedure effected on the skin of a subject. Said tip or delivery member is preferably disposable. Said tubing is preferably a chemically stable tubing, preferably flexible along most of its length, possibly composed of several sections, wherein the section in contact with the heat exchanger may have higher thermal conductivity than the remaining parts, and the part within the liquid pump may have higher flexibility and mechanical stability. The heat exchanger is preferable metal, such as comprising copper or aluminum. Said liquid pump preferably controls said flowrate with an accuracy of at least 0.01 ml/sec, said tubing preferably comprises a disposable silicone tube, said exchanger preferably comprises aluminum plates provided with a coiled channel for accommodating said tubing and controlling the liquid temperature with an accuracy of at least 0.5° C., such as at least 0.3° C.; the channel may be realized by two metallic plates sandwiching a tube in a serpentine groove machined into the plates.

Said microprocessor stores data and calculates the heat loss along the tubing length, processes the liquid temperatures in said container and exchanger, as well as said working temperature, and activates the Peltier module to cool or heat the exchanger. The device or system of the invention is in one aspect employed in eradicating tattoos on the skin, and may further comprise at least some of the following elements: a needles array for puncturing the skin, an actuator designed to repeatedly thrust the needles at a predetermined frequency into predetermined depth of the skin, a suction means for removing the liquid, and ingredients, after its contact with epidermis and/or dermis (used liquid), and a sealing means preventing an escape or spill of the liquid outside the device. The device according to the invention for removing tattoos and employing a working liquid with a precise temperature control may further comprise an item selected from the group consisting of a means for eventual rinsing the needles with fresh sterile liquid, a means providing separation between the fresh and used liquid, a trigger activating the actuator and the liquid pump, an external or internal power source, a disposal means for disposing of the used liquid, and a cylindrically or conically shaped disposable tip or delivery member in contact with the skin through which the liquid with precisely controlled temperature and flow rate is delivered to the skin vicinity. The device or system of the invention may comprise elements or features employed in the skin puncturing apparatus described in WO 2012/140643 of the same inventors.

In a preferred embodiment of the invention, provided is an apparatus for removing large tattoos, comprising pigments of any color. The sterile liquid delivered to the skin and acting in the skin (the working liquid) in the system of the device and system according to the invention has a precisely controlled temperature and flowrate. The active liquid may comprise an item selected from the group consisting of solvents, salts, peroxides, organic acids, structural proteins and other proteins, enzymes such as enzymes selected from hydrolases, complexing agents, and natural materials such as plant extracts. The active liquid may comprise components selected by a skilled person for specific needs, including toxins, polysaccharides, immune-components, etc. For example, said polysaccharide may be hyaluronic acid, said structural protein may be collagen, etc. Various components may include, for example, distilled water, DMSO, EDTA, collagenase, hyaluronidase, papain, bromelain, hypertonic saline, salicylic acid, hydrogen peroxide, etc. The liquid is usually an aqueous liquid, such as an aqueous solution. In a preferred embodiment, the working liquid comprises salicylic acid or a salt thereof in water.

The invention relates to an aesthetic or cosmetic procedure for treating the skin employing a liquid delivered to the skin with precisely controlled temperature and flowrate, comprising i) providing a heat exchanger cooled and heated by a heat pump, such as a solid-state Peltier module, or another heat exchange system, with a precision of at least 0.5° C., ii) pumping said liquid through said exchanger by a liquid pump, such as a peristaltic pump or other suitable pump with a precision of at least 0.01 ml/s, and iii) delivering the liquid to the skin via a disposable tip in the vicinity of a needles array. In a preferred arrangement, the procedure according to the invention comprises a step of i) repeatedly thrusting needles at a predetermined frequency into predetermined depth of the skin; and ii) sucking the liquid after its contact with epidermis and/or dermis and disposing of it. The procedure is preferably employed for removing large tattoos of any color.

In one embodiment of the invention, the procedure further comprises a step of preparing an aqueous liquid, for example an aqueous solution, comprising an active agent, for example salicylic acid or a salt thereof, and pumping the liquid at a precisely controlled temperature and flowrate to the skin, without perforating the skin or without immediately perforating the skin.

### BRIEF DESCRIPTION OF THE DRAWINGS

The above and other characteristics and advantages of the invention will be more readily apparent through the following examples, and with reference to the appended drawings, wherein:

FIG. 1. is a scheme outlining a device and a system according to one embodiment of the invention; the numbers represent: 1—tubing, 2—tubing heat sink, 3—thermocouple, 4—inlet port, 5—needles area, 6—Peltier module, 7—fan heat sink, 8—fan, 9—set point, 10—comparator, 11—PWM, 12—H-bridge;

FIG. 2. schematically shows an equipment for removing a tattoo according to WO 2012/140643 with a device for controlling the temperature of the working liquid according to the present invention; the working liquid flows from a stock container located at ambient temperature to a precise peristaltic pump which delivers the working liquid with a high precision even at flow rates of 1-5 ml/min to a pistol-shaped apparatus provided with a temperature sensor, heat exchanger through which the working liquid flows via silicon tubing, and a solid-state Peltier module with a fan, the tubing delivering the working liquid with precisely adjusted temperature to the skin via a disposable tip;

FIG. 3. shows the pistol-shaped apparatus of FIG. 2 with an opened metal heat exchange part for accommodating a coiled tubing;

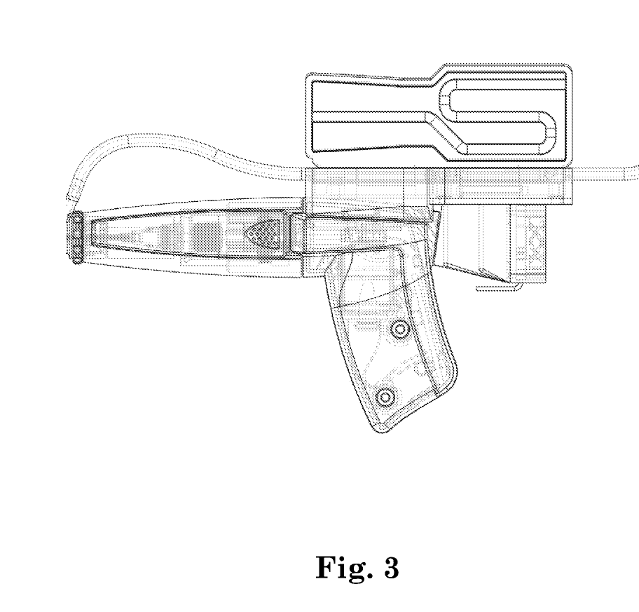

FIG. 4. is a perspective view (from above) of the pistol-shaped apparatus of FIG. 3;

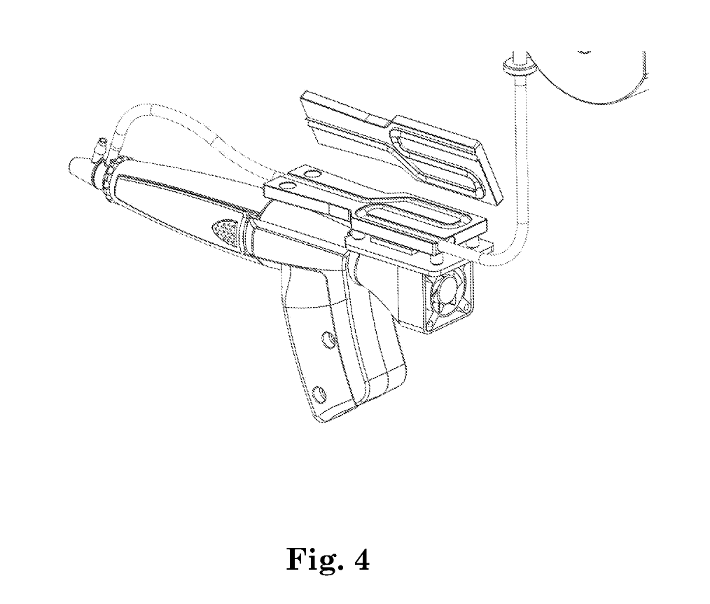

FIG. 5. is a detail of the rear part of the pistol-shaped apparatus of FIG. 3 in a perspective view (from below), with tubing closed in the heat exchanger;

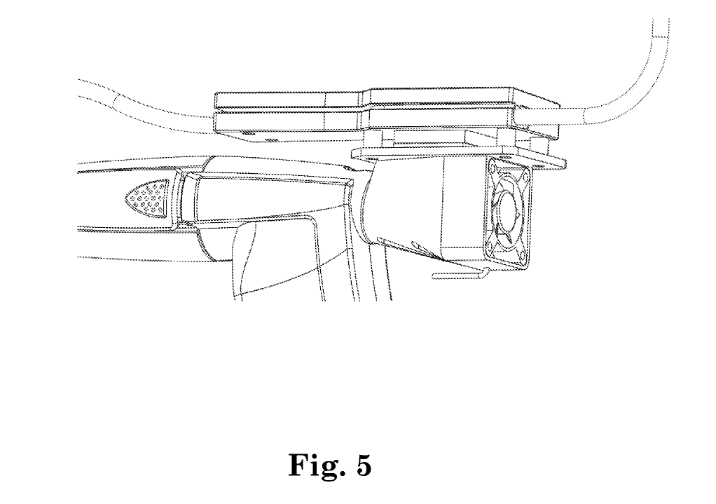

FIG. 6. is a detail of the rear part of the pistol-shaped apparatus of FIG. 3 in a perspective view (from above), with exploded view of the heat exchanger;

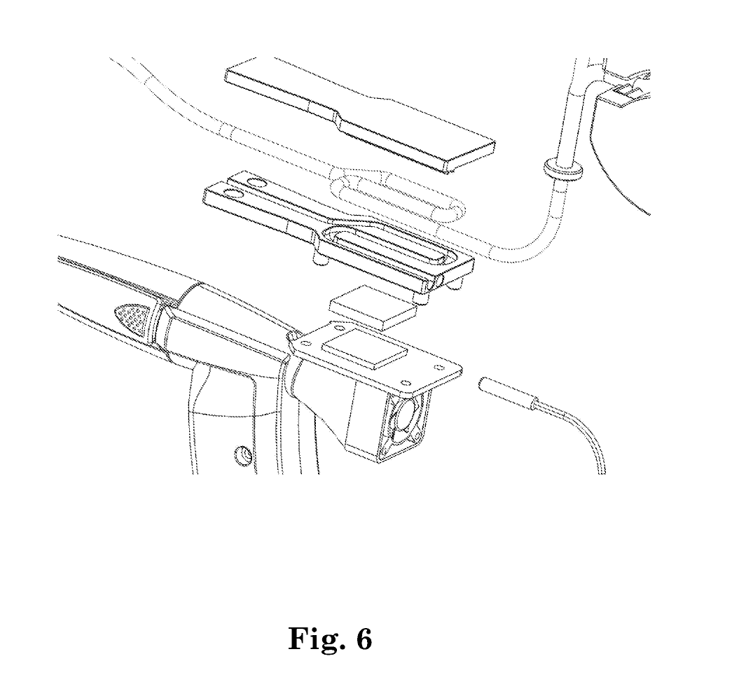

FIG. 7. is a rear view of the pistol-shaped apparatus of FIG. 3, with the fan for cooling the Peltier module; and

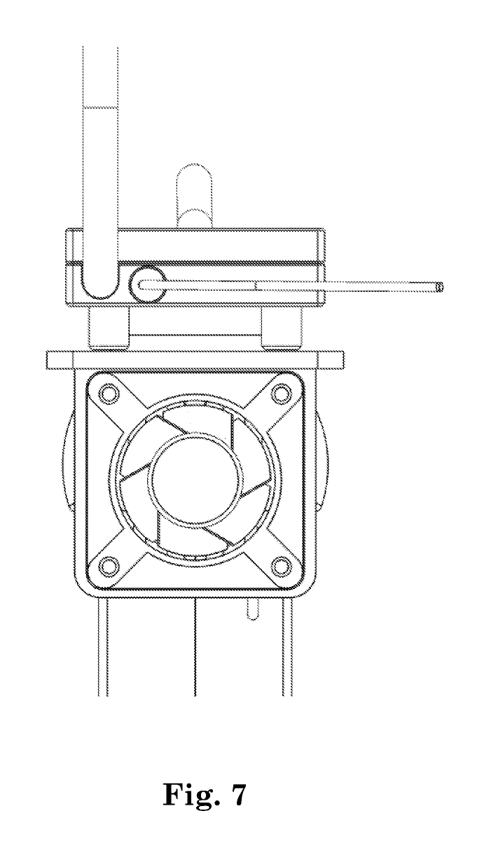

FIG. 8. is a detail of the peristaltic pump of the equipment in FIG. 2.

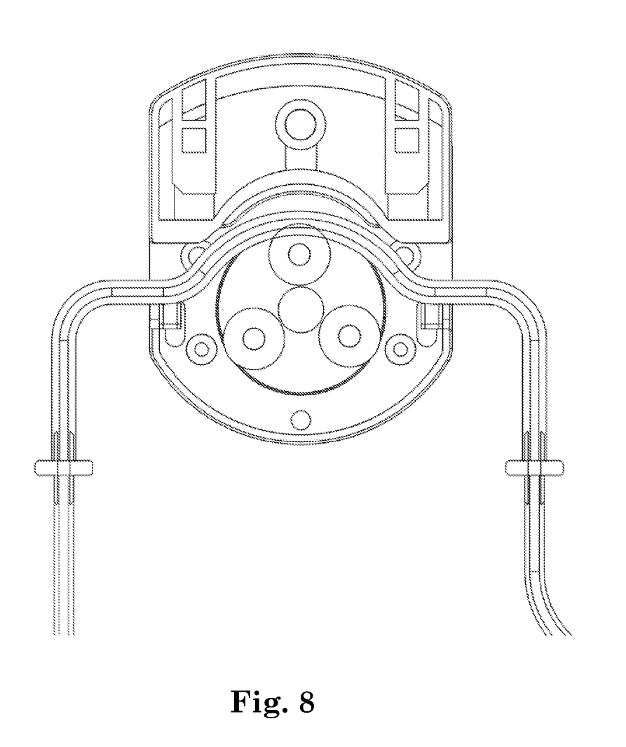

### DETAILED DESCRIPTION OF THE INVENTION

It has now been found that the temperature of active liquids used in tattoo removal or various aesthetic treatments, for example liquids delivered onto the skin by a pistol-shaped apparatus for tattoo removal disclosed in WO 2012/140643, can be efficiently controlled by a system comprising precise peristaltic pump connected with said apparatus, thermocouple for sensing temperature in the apparatus, and a solid-state Peltier module integrated in the apparatus, thereby improving the efficiency of the method.

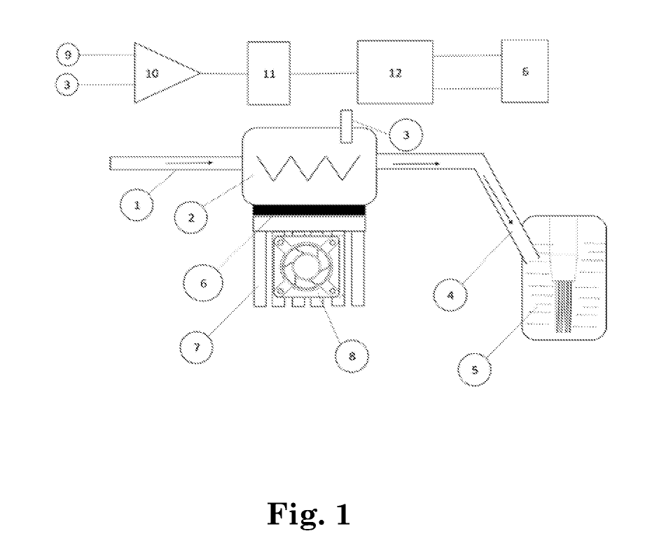

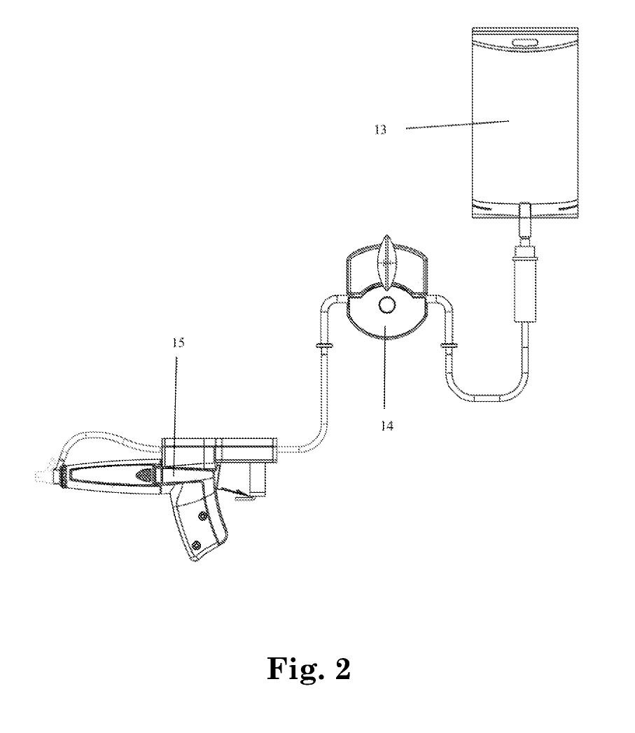

In one aspect, the invention provides a system for aesthetic, cosmetic or curative delivery of liquids onto the skin, comprising a needles array and a delivery means which supplies the liquid in a predetermined flow rate (typically in a range of about 1-10 ml/min) and temperature (typically in a range of 5-40° C.). FIG. 1 and FIG. 2 show a system in some embodiments of the invention, the main elements being briefly described as follows:
A small infusion bag (13) containing a liquid stock, substantially at room temperature, supplies the liquid to a hand-piece via tube (1).
The delivery is controlled by a precise peristaltic pump (14) capable of metering the flow to an accuracy of typically 0.01 ml/sec and delivering a flow of approximately 0.05 ml/sec.
A disposable silicone tube (1), typically 4 mm OD, which conveys the liquid from the pump through the heat exchanger (2) and then to the needle area (5), via port (4).
A heat exchanger (2) which heats or cools the liquid to a desired temperature (typically in the 5-40° C. range). The heat exchanger is designed to enable quick replacement of the single use silicone tube between treatments and yet provide good thermal contact with the liquid.
A solid-state Peltier module (6) in good thermal contact with the heat exchanger (2) on one side and with a fan (8) and cooling heat sink (7) on the other side.
An assembly (5) consisting of the needles, suction shroud and actuator designed to oscillate the needles at a predetermined frequency, irrigate the skin in the vicinity of the needle and eject the spent solution plus skin fragments (“used liquid”) by vacuuming the shroud (sealed) space.
A microprocessor receiving the temperature from thermocouple (3), calculating the heat loss between the thermocouple and the skin, and activating the Peltier module (6), whereby ensuring the final predetermined temperature of the liquid when it comes into contact with the skin. This includes a feedback control system consisting of said temperature sensor (3), typically thermocouple of type K or T, embedded in one of the metallic plates of (2), supplying an input signal to a controller and comparator (10) which also contains the set point (9). By proportional control, polarity changeover and PWM (pulse width modulation, 11) output to the voltage applied to the Peltier (6) via H-Bridge (12), the desired temperature is maintained (in both heating and cooling) to an accuracy of±0.3° C.

The following thermal aspects are taken into consideration. The system is required to supply energy to bring the solution from RT to a maximum of 40° C. at a maximum flow rate of 0.05 ml/sec. This requires a heat input of approximately 3 W (Q=m*Cp*ΔT, where Q is heat flowrate, m is mass flowrate, Cp is specific heat capacity, and ΔT is temperature change). The 3 W heat input must be transferred to the liquid in a volume and weight which can be conveniently housed in a hand-piece and which enables simple and quick replacement of the fluid carrying duct. The hand-piece may acquire the pistol-shape of the skin puncturing apparatus for use in eradicating tattoos according to WO 2012/140643 of the same inventors. It is intended to perform this by means of two metallic plates (typically aluminum, 3-4 mm thick) which sandwich the silicone tube in a serpentine groove machined into the plates. One of the plates is heated or cooled by the Peltier element. In another embodiment, the serpentine part of the tube can be replaced by a small infusion bag which is trapped between the two metallic plates. The lower plate is heated or cooled by the Peltier and thus controls the temperature of the liquid. By employing sufficient contact area to minimize the thermal resistance between the plate and liquid, the desired liquid temperature can be held to an accuracy of typically±0.3° C. using feedback control from the metallic plate temperature alone. The desired liquid working temperature at the work site, near the needles or the skin, will usually not be significantly different from the liquid temperature in the plate, as a result of the low level of heat transfer from the exposed part of the tube, as our tests showed, and calculations confirmed; however, any losses may be compensated for by slightly increasing the temperature in the hand-piece (indicated by thermocouple 3) relatively to the desired final working temperature (9) (experiments indicated typically not more than 0.1-0.2 W losses between 40° C. and 25° C.).

The device according to the invention may, in one embodiment, be employed within an apparatus for non-surgical eradication of a tattoo from an area of skin as disclosed in WO 2012/140643. The apparatus of said prior art may be shaped like a pistol comprising handle section and barrel section, wherein the elements of the instant device, as seen in

FIG. 1, nos. 2, 3, and 6-8 may be comprised in said handle section, and element no. 4 may be comprised in said barrel section of the pistol-shaped apparatus, for example such as member 15 in FIG. 2.

In one embodiment of the invention, the system for controlling the temperature of a liquid delivered to the skin is integrated with an apparatus for efficiently removing tattoos from the skin, possibly even a multi-color large tattoo, for example an apparatus according to WO 2012/140643. A system or a device according to the invention, integrated into various apparatuses, may comprise elements selected from the group consisting of
a motor for moving a needles-array and the array of needles to be pushed through the outer layer of skin into the dermis and withdrawn from the skin,
an external or internal power source,
a means for pressing the liquid outlet against the skin so that a hermetic seal is created isolating the interior of the barrel section from the outside,
a means for sucking the used liquid away from the skin (the term “used liquid” relates to the liquid containing cellular fluid and tattoo pigments),
a means for eventual rinsing the needles with fresh liquid,
a means providing separation between the clean and the used liquid, wherein the active liquid preferably comprises water, and further may comprise other solvents, salts, buffers, organic acids, complexing agents, reagents such as including oxidizers, enzymes such as hydrolases, naturally derived components such as plant extracts, and others,
a trigger activating the motor of the prior art apparatus and the peristaltic pump of the invention,
a disposable, cylindrically or conically shaped tip or delivery member in contact with the skin through which the liquid, with precisely controlled temperature and flow rate, is delivered onto and into the skin,
a storage means of the fresh liquid, for example cooled or placed at ambient temperature, and
a disposal means for disposing of the used liquid.

The system and the device of the invention preferably comprise a microprocessor activating a heat pump, such as a Peltier module, but also other heat exchanging arrangement can be conveniently applied by the user, such as a plastic bag similar to infusion bag which is sandwiched between two suitable plates, wherein the microprocessor comprises data characterizing the system, including the desired flow rate of the liquid to be delivered onto or into the skin, tubing length and diameter of all tubing sections, heat transfer parameters of the involved materials, desired temperature of the liquid on the skin (working temperature), etc., wherein the microprocessor receives also the sensor temperature (from the thermocouple) and the storage temperature of the liquid stock. Of course, when providing the data, a skilled person will employ known relations for heat transfer, as well as for temperature effects on diffusion, including Stokes-Einstein equation, based on the famous Einstein's work on Brownian motion from 1905.

The prior art liquid delivery apparatuses, particularly the apparatus for eradicating tattoos, lacked the system for controlling the temperature of a liquid being stored at ambient or storage temperature and preferably working on skin at a higher temperature such as 40° C. The invention provides the temperature control for the liquid delivered through a disposable tip or other delivery member by employing a suitable heat pump like Peltier element, with a precise pump, and a simple processor. The system advantageously combines low thermal inertia tubing, heater aluminum plate exhibiting very good thermal contact with the tubing, processor providing accurate calculations, and elements with rapid response and accurate control. The administration of the active solutions onto the skin and into the dermis under isothermal condition improves the biochemical reactivity of the agents and their bonding to the pigments particles, thereby allowing for their eventual transport to an extracellular ambience and removal from the skin. In another aspect, the temperature control leading to a significant reduction of the thermal excursion can have a positive repercussion on the inflammatory induced response. Controlling the temperature contributes to the efficiency of pigments removal and minimizes the potential side effects.

The invention will be further described and illustrated in the following examples.

### EXAMPLES

Practically, tube wall heat transfer was assessed while using typical values for total resistance, heater plate to liquid, of about 3 C/W, the overall heat transfer being (ΔT/total resistance=3.2 W) for a heating plate temperature of 40° C. and solution temp of 30° C. This heat load can conveniently be supplied by a Peltier element of approximately 30×30 mm (such as Ferrotec 9501-127-030) operating at 8V, 3-4 A in contact with a heat-sink/fan assembly. Typical heat-sink thermal resistances are 0.2-0.4 C/W for a 40×40 mm footprint. This is adequate to maintain the Peltier at its normal operating temperature. The needed parameters can be easily obtained, including film coefficient of heat transfer, utilizing Reynolds number, Nusselt number, etc., the latter being possibly about 3.6 under usual conditions, providing film coefficient of about 1000 W/(m²·° C.) and liquid side resistance of 1.49° C./W. Tube wall heat transfer is approximately proportional to the tube wall thickness divided by the product of the tube conductivity and the wetted area available for heat transfer; typical values yield a tube wall resistance of 1.59° C./W. An estimate of 0.001° C./W is made for tube to plate interfacial resistance, based on known parameters for soft rubber and assuming good contact. The total resistance may be assessed, for the sake of example, as 1.49+1.59+0.001=3 C/W. Overall heat transferred may be assessed as (Tplate−Tliquid)/total resistance=3.2 W for a plate temperature of 40° C. and a solution temperature of 30° C. This heat load can conveniently be supplied by a Peltier element of approximately 30×30 mm (e.g. Ferrotec 9501-127-030) operating at 8 V and 3-4 A in contact with a heat-sink/fan assembly. Typical heat-sink thermal resistance is about 0.2 C/W for a 40×40 mm footprint. This is adequate to maintain the Peltier at its normal operating temperature.

While this invention has been described in terms of some specific examples, many modifications and variations are possible. It is therefore understood that within the scope of the appended claims, the invention may be realized otherwise than as specifically described.

1. A device for controlling the temperature of a liquid to be delivered onto or into the skin of a subject, comprising
i) a container for storing said liquid at a temperature between above its freezing temperature and ambient temperature;
ii) a liquid pump, such as a peristaltic pump conferring a desired flowrate to said liquid;
iii) a heat exchanger which heats or cools the liquid to a working temperature up to 60° C., usually between 5° C. and 40° C.;
iv) a temperature sensor suitable to detect the temperature of the liquid at the outlet from said heat exchanger;
v) a heat pump, such as a solid-state Peltier module in thermal contact with said heat exchanger on one side, and optionally with a fan and cooling heat sink on the other side;
vi) a disposable or reusable tip suitable to be placed in contact with said skin and to irrigate the skin with said liquid;
vii) a tubing suitable to deliver said liquid from said container to said tip; and
viii) a microprocessor or a control system suitable for receiving a temperature reading from said sensor and the flow rate of said liquid pump and for activating said heat pump such that the liquid delivered to said tip is delivered at a desired temperature.

2. The device of claim 1, wherein said peristaltic pump is suitable to control said flowrate with an accuracy of at least 0.01 ml/sec, said tubing comprises a disposable silicone tube, said exchanger comprises aluminum plates provided with a coiled channel for accommodating said tubing and for controlling the liquid temperature with an accuracy of at least 0.3° C.

3. The device of claim 1, wherein said microprocessor is configured to calculate the heat loss along the tubing length, to process the liquid temperatures in said container and exchanger, and said working temperature, and to activate the Peltier module to cool or heat the exchanger.

4. The device of claim 1, further comprising a needles array for puncturing the skin, an actuator designed to repeatedly thrust the needles at a selected frequency into a selected depth of the skin, suction means for removing used liquid after its contact with epidermis and/or dermis, and sealing means preventing an escape or spill of the liquid outside the device.

5. The device of claim 4, further comprising an item selected from the group consisting of: means for eventual rinsing the needles with fresh liquid, means providing separation between the fresh and used liquid, a trigger activating the actuator and the peristaltic pump, an external or internal power source, disposal means for disposing of the used liquid, and a cylindrically or conically shaped disposable tip or delivery member in contact with the skin through which the liquid with precisely controlled temperature and flow rate can be delivered to the skin vicinity.

6. The device of claim 1, wherein its components are suitable for removing a tattoo or pigments from the skin.

7. A cosmetic or aesthetic procedure for treating the skin employing a liquid delivered to the skin with precisely controlled temperature and flowrate, comprising:
i) providing a container for storing said liquid at a temperature between above its freezing temperature and ambient temperature;
ii) providing a peristaltic pump conferring a desired flowrate to said liquid;
iii) providing a heat exchanger which heats or cools the liquid to a working temperature of up to 60° C., usually between 5° C. and 40° C.;
iv) sensing a liquid temperature, using a temperature sensor suitable to detect the temperature of the liquid at the outlet from said heat exchanger;
v) placing a solid-state Peltier module in thermal contact with said heat exchanger on one side and with a fan and cooling heat sink on the other side;
vi) placing a disposable tip in contact with said skin thereby to irrigate the skin with said liquid;
vii) providing a tubing suitable to deliver said liquid from said container to said tip; and
viii) activating said Peltier module such that the liquid delivered to said tip is delivered at a desired temperature, using a microprocessor suitable for receiving a temperature reading from said sensor and the flow rate of said peristaltic pump.

8. The procedure of claim 7, further comprising
i) providing a needles array for puncturing the skin and repeatedly thrusting said needles at a selected frequency into a desired depth of the skin; and
ii) sucking the liquid after its contact with epidermis and/or dermis and disposing of it.

9. The procedure of claim 7, comprising removing large tattoos of any color.

10. The procedure of claim 7, further comprising providing an aqueous liquid with an active agent, and pumping it at a precisely controlled temperature and flowrate to the skin without perforating the skin.
